(19) United States
(12) Patent Application Publication
(10) Pub. No.: US 2004/0248202 A1
(43) Pub. Date: Dec. 9, 2004

(54) Bacteriorhodopsin/G protein-coupled receptor chimeras

(75) Inventors: Arnold E. Ruoho, Madison, WI (US); Michael K. Sievert, Madison, WI (US); Mark P. Krebs, Madison, WI (US); Andrew H. Geiser, Madison, WI (US)

Correspondence Address: MICHAEL BEST & FRIEDRICH, LLP, ONE SOUTH PINCKNEY STREET, P O BOX 1806, MADISON, WI, 53701

(73) Assignee: Wisconsin Alumni Research Foundation, Madison, WI

(21) Appl. No.: 10/688,221

(22) Filed: Oct. 16, 2003

Related U.S. Application Data
(63) Continuation of application No. 09/389,835, filed on Sep. 3, 1999, now abandoned.
(60) Provisional application No. 60/098,950, filed on Sep. 3, 1998.

(51) Int. Cl. G01N 33/53; C07H 21/04; C12P 21/04; C12N 9/00; C12N 1/21; C07K 14/705

(52) U.S. Cl. 435/7.1; 435/69.7; 435/320.1; 435/252.3; 530/350; 536/23.5

(57) ABSTRACT

Disclosed is a chimeric protein comprising a bacteriorhodopsin polypeptide sequence and a G protein-coupled receptor sequence. Also disclosed is a genetic construct comprising encoding a chimeric bacteriorhodopsin polypeptide sequence and a G protein-coupled receptor sequence. The chimeric protein of the present invention can be used to evaluate various agents for the ability to interact with loop 3 of a G protein-coupled receptor.

### CROSS-REFERENCE TO RELATED APPLICATION

This application claims priority to U.S. Provisional Application Ser. No. 60/098,950, filed Sep. 3, 1998 which is incorporated by reference herein.

### STATEMENT REGARDING FEDERALLY SPONSORED RESEARCH OR DEVELOPMENT

This invention was supported by United States government funds in the form of grant NIH GM33138, awarded by the National Institutes of Health.

### BACKGROUND OF THE INVENTION

Signal transduction is an exquisite regulatory system that affords rapid adjustment of intracellular functions and activities in response to changes in the extracellular environment. In multicellular eukaryotic organisms, signal transduction allows communication between various specialized cell types.

Guanine nucleotide-binding (G) protein-coupled receptors constitute a superfamily of receptors that are involved in regulating the function of virtually every cell in the human body. Currently, more than 250 types of G protein-coupled receptors have been identified, including beta-adrenergic, serotonin, muscarinic acetylcholine, tachykinin, prostaglandin, and rhodopsin receptors. It is believed that this superfamily may contain thousands of members.

G protein-coupled receptors are heptahelical proteins with seven transmembrane domains, three extracellular loops, and three intracellular loops, with the amino (N) terminus oriented extracellularly and the carboxy (C) terminus oriented intracellularly. The intracellular loop proximal to the C-terminus (loop 3) is highly variable in length and is believed to confer specificity of interaction with G proteins.

When G protein-coupled receptors bind to specific ligands at the extracellular surface of the cell membrane, a conformational change in the G protein receptor is believed to occur. Ligand binding allows intracellular loop 3 to associate with an intracellular G protein. The interaction between the G protein-coupled receptor and the G protein triggers a cascade of intracellular events mediated by various downstream effectors, such as adenylate cyclase.

G protein-coupled receptors are critically important in regulating normal cellular functions and in maintaining health. These receptors also play an important role in the design and development of drug treatments for various diseases. Drugs that are either agonistic or antagonistic for ligands that bind to particular receptors have been developed to alter intracellular activities. For example, β blockers are a class of β adrenergic antagonists that are commonly used in the treatment of high blood pressure.

A number of diseases have been linked to mutations that interfere with the ability of a G protein-coupled receptor to bind to extracellular ligands. Diseases known to be caused by G-protein-coupled receptor mutations include, for example, retinitis pigmentosa, color blindness, nephrogenic diabetes insipidus, and hyperfunctioning thyroid adenomas. These diseases, which are either heritable or caused by somatic mutations, are refractory to treatment using agonists or antagonists. It is expected that the number of diseases attributable to mutations in G protein-coupled receptors will continue to grow as more is learned about this important family of receptors.

Several serious diseases are associated with the uncoupling of ligand binding to G protein-coupled receptors and activation of the signaling pathway. In other words, the signaling pathway is constitutively activated in an agonist-independent manner. For example, Kaposis sarcoma-associated herpesvirus (KSHV/HHV8) encodes a G-protein coupled receptor that stimulates signaling pathways constitutively to induce transformation and angiogenesis in KSHV-mediated oncogenesis. (Bais et al., Nature 391:86, 1998).

Agonists or antagonists of ligands that normally bind to G protein-coupled receptors are not effective in treating diseases caused by mutations that reduce or prevent ligand binding to G protein-coupled receptors, or which result from the constitutive activation of signaling pathways in an agonist-independent manner. Treatment of these types of diseases depends upon the development of pharmaceuticals capable of altering the activation of signaling pathways at a different level of control. We propose that this may be achieved by enhancing or reducing G protein interaction with a G protein-coupled receptor, thereby altering the activity of G proteins.

Progress in elucidating the mechanism by which G proteins function in signal transduction has been impeded by difficulties associated with isolating G protein-coupled receptors in intact, functional form and in quantities sufficient to allow comprehensive studies to be conducted.

What is needed in the art is a method by which receptor proteins having a domain associated with G-protein activation can be isolated in quantities sufficient to allow screening of potential therapeutic agents.

### BRIEF SUMMARY OF THE INVENTION

The development of efficacious drugs for treating diseases caused by errors in signaling pathways involving G protein-coupled receptors would be facilitated by a method of obtaining sufficient amounts of protein comprising relevant G protein-coupled receptor sequences. The ability to obtain a chimeric protein comprising at least a portion of intracellular loop 3 will facilitate screening of potential therapeutics for interaction with this critical domain.

The present invention includes a chimeric protein comprising a bacteriorhodopsin polypeptide sequence and a G protein-coupled receptor polypeptide sequence. Preferably, the protein comprises a bacteriorhodopsin scaffolding in which the amino acid sequence specifying intracellular loop 3 of bacteriorhodopsin has been replaced by an amino acid sequence specifying at least a portion of intracellular loop 3 of a G protein-coupled receptor.

Another aspect of the present invention is a genetic construct comprising a nucleotide sequence that encodes a bacteriorhodopsin/G-protein coupled receptor chimeric protein operably connected to a promoter.

Yet another aspect of the present invention is a method of preparing a bacteriorhodopsin/G protein-coupled receptor chimeric protein comprising the steps of transforming an archaebacterium with a genetic construct comprising a nucleotide sequence that encodes a bacteriorhodopsin/G protein-coupled receptor chimeric protein, the nucleic acid sequence expressible in the archaebacterium host, culturing the archaebacterium under suitable conditions of growth, and allowing expression of the chimeric protein.

It is an object of the present invention to provide a bacteriorhodopsin/G protein-coupled receptor protein chimeric protein that may be used in further research into the role of G protein-coupled receptors in signal transduction and in drug development assays.

It is an advantage of the present invention that intact bacteriorhodopsin/G protein-coupled receptor chimeric protein can be isolated in quantities sufficient to be used in high throughput assays of potential therapeutics, as well as to obtain information about the structure of the replacement loop.

Other objects, features, and advantages of the present invention will be apparent upon review of the specification and claims.

### DETAILED DESCRIPTION OF THE INVENTION

The method of the present invention makes it possible to obtain relatively large quantities of a chimeric, membrane-associated fusion protein that primarily comprises largely bacteriorhodopsin amino acid sequence, in which an amino acid sequence that specifies a portion of the bacteriorhodopsin protein has been replaced by an amino acid sequence for the structurally analogous region from a G-protein coupled receptor. The availability of chimeric bacteriorhodopsin/G-protein coupled receptor fusion proteins will facilitate studies designed to assess the role of various domains of G-protein coupled receptors in signal transduction, and facilitate identification of potential therapeutic agents that are capable of interacting with the G protein-coupled receptor so as to alter signal transduction.

Bacteriorhodopsin is a membrane protein found in halophilic archaebacteria. Bacteriorhodopsin is structurally very similar to the G protein-coupled receptors, in that it is a heptahelical protein with seven transmembrane domains, three extracellular loops, and three intracellular loops, with the amino (N) terminus oriented extracellularly and the carboxy (C) terminus oriented intracellularly. However, bacteriorhodopsin is functionally distinct from the G protein-coupled receptors in that it functions as a proton “pump” in the transduction of light energy to chemical energy. Bacteriorhodopsin has been extremely well characterized, in part because the protein forms a crystal structure in its native membrane environment, which facilitates purification and characterization.

U.S. Pat. No. 5,641,650, incorporated by reference herein, discloses a method for producing a heterologous polypeptide by expressing a heterologous DNA sequence under the control of the transcriptional and translational regulatory sequences from a bacteriorhodopsin gene in halobacterium. Disclosed examples of heterologous proteins thus expressed include G protein-coupled receptors and G protein-coupled receptors lacking intracellular loop three.

Like bacteriorhodopsin, the chimeric fusion proteins produced by the method of the present invention are directed toward the membrane and are able to bind retinal, which suggests that these chimeras may have a crystal lattice structure comparable to that of bacteriorhodopsin. Preliminary comparative data obtained by ultraviolet and visible absorbance spectrophotometry (UV/VIS), circulator dichromism (CD), and sucrose density gradient isolation suggest that the chimeras may have a crystal structure similar to that of bacteriorhodopsin. Chimeric fusion proteins in which discrete portions of bacteriorhodopsin have been replaced with a specific domain from G protein-coupled receptor proteins will allow further investigation into the roles specific domains play in signal transduction.

Large quantities of G protein-coupled receptor intracellular loop 3 in a lattice structure were obtained in the form of chimeric fusion proteins in which the third loop of the bacteriorhodopsin protein was replaced with the third loop of any one of several G protein receptors.

A bacteriorhodopsin DNA fragment comprising the bacteriorhodopsin gene from Halobacterium salinarium was used to construct the chimeras as described below. The sequences of the DNA fragment and the putative amino acid sequence of bacteriorhoposin are shown in SEQ ID NO: 1 and SEQ ID NO:2, respectively. The coding sequence for bacteriorhodopsin is found at base 394-1182 of sequence ID NO:1. Amino acid residues 1-13 of SEQ ID NO:2 constitute a leader sequence that is cleaved to form the mature protein (amino acids 14-262 of SEQ ID NO:2). It is expected that any bacteriorhodopsin gene from any archaebacterial species could be successfully employed in the practice of this invention. A bacteriorhodopsin gene of known sequence is preferable, because the sequence information facilitates selection of suitable restriction enzymes and design of oligonucleotide primers that can be used to construct a sequence encoding a chimeric protein.

Halobacterium salinarium strain MPK40, which was engineered to delete the bacteriorhodopsin gene (Krebs, et al. Proc. Natl. Acad. Sci. USA, 90:3986-3990, 1993; incorporated by reference herein) was used as a host strain for obtaining stable transformants comprising chimeric protein coding sequences integrated into the archaebacterial chromosome. In contrast to wild type Halobacterium strains, which produce purple colonies, MPK40 produce orange colonies. Transformants of MPK40 containing a chimeric bacteriorhodopsin/G protein-coupled receptor gene were used to express the chimeric proteins.

It is expected that any other suitable archaebacterial species or strain may serve as a host for expressing the chimeric proteins. A suitable archaebacterial species or strain is one that is capable of expressing a chimeric bacteriorhodopsin/G protein-coupled receptor gene to produce a chimeric bacteriorhodopsin/G protein-coupled receptor fusion protein. Preferably, the strain lacks a bacteriorhodopsin gene. It is well within the ability of one of skill in the art to genetically engineer an archaebacterial strain to delete a particular gene, such as the bacteriorhodopsin gene.

Plasmid pMPK85, which was used to develop the transformants, has an insertion sequence (ISH1) (SEQ ID NO:45) that allows integration of the bacteriorhodopsin/G protein-coupled receptor gene into the archaebacterial chromosome. It is envisioned that other IS elements may be useful in obtaining stable integration of a bacteriorhodopsin/G protein-coupled receptor gene.

In designing the chimeric proteins of the present invention, in vitro mutagenesis was used to introduce a unique BstXI restriction site in transmembrane domain six. The native bacteriorhodopsin loop 3 coding region was removed by digestion with restriction endonucleases BstXI and BsrGI, which digests the bacteriorhodopsin (BOP) gene at a unique site in the coding region for transdomain 5. One of skill in the art can appreciate that similar constructs could be prepared using engineered or native restriction sites other than BstXI and BsrGI. In addition to the transdomain 6 mutation, a Bsu36I site was created in transmembrane domain 7 using in vitro mutagenesis. This restriction site may be used in constructing chimeras in which the carboxy terminus of bacteriorhodopsin is replaced with the carboxy terminus of a G protein-coupled receptor.

The mutations created in the bacteriorhodopsin gene are silent, in that these mutations do not alter the amino acid sequence of the protein expression product. In designing the chimeric proteins of the present invention, one of skill in the art would recognize that an alteration in the primary sequence of a transmembrane domain could result in perturbation of the higher order protein structure. It should be appreciated that it may be possible to make certain conservative substitutions in the amino acid sequence without affecting the secondary or tertiary structures.

In the examples below, chimeric proteins were constructed in which a portion of intracellular loop 3 of bacteriorhodopsin was replaced with the structurally analogous intracellular loop 3 region from bovine rhodopsin, adrenocorticotrophic hormone receptor, adenosine A1 receptor, or human beta2 adrenergic receptor. It is reasonable to expect that the present invention could be successfully practiced using a loop 3 region from any G protein-coupled receptor.

By “an intracellular loop 3 region” it is meant an amino acid sequence that includes at least a portion of an amino acid sequence corresponding to the amino acid sequence that forms intracellular loop three of a G protein-coupled receptor. Preferably, the intracellular loop three region is one that is able to alter the rate of GTP-GDP exchange from a G protein in an in vitro exchange assay.

The oligonucleotide sequences encoding G protein-coupled receptor sequences and used in the construction of the bacteriorhodopsin G protein-coupled receptor chimeras are preferably designed to optimize codon usage in Halobacterium salinarium so as to prevent pausing during translation and to maximize protein yields. However, it is expected that minor variations in sequence associated with nucleotide additions, deletions, and mutations, whether naturally occurring or introduced in vitro may not affect the expression of the chimeric protein. The scope of the present invention is intended to encompass minor variations in the chimeric protein sequence.

The chimeric proteins described below contain substantially all of the amino acid sequence of the wild type bacteriorhodopsin, with the exception for the deletion of intracellular loop 3 amino acid 171 to amino acid 179 of SEQ ID NO:2. The region corresponding to amino acids 171-179 of SEQ ID NO:2 was replaced with an intracellular loop 3 sequence from one of three G protein-coupled receptors. In each case, the G protein-coupled receptor loop 3 region was inserted between the threonine residue at position 170 of SEQ ID NO:2 (amino acid residue 157 of the mature bacteriorhodopsin) and the valine residue at position 180 of SEQ ID NO:2 (amino acid residue 167 of the mature bacteriorhodopsin).

It should be appreciated that it other portions of bacteriorhodopsin protein may be replaced with the corresponding structurally analogous sequence from a G protein-coupled receptor. By a “structurally analogous sequence” it is meant a sequence that forms a region of the G protein-coupled receptor corresponding to the bacteriorhodopsin region to be replaced (e.g., an intracellular or extracellular loop, the N-terminal or C-terminal region, or portions thereof). One wishing to substitute a portion of another region or regions of bacteriorhodopsin sequences with sequences from G protein-coupled receptors could readily do so, using the teachings of the present invention and standard molecular biology techniques. By way of example, it may be advantageous to create a bacteriorhodopsin/G protein-coupled chimera comprising the intracellular loop 3 region and the C-terminal region of a G protein-coupled receptor. This could be accomplished through the proper selection of restriction enzymes and primers, using the teachings and guidance provided herein.

The intracellular loop 3 of G protein-coupled receptors is involved in promoting GTP-GDP exchange in G-proteins. The functionality of six different chimeric proteins in which the bacteriorhodopsin loop three region had been replaced with various regions of bovine rhodopsin intracellular loop three was evaluated by measuring the rate of GTP-GDP exchange using transducin. Some, but not all, tested chimeric proteins were found to enhance GTP-GDP exchange relative to basal levels of exchange. This effect was inhibited by the presence of a high affinity analog of a peptide based on the C-terminus of the transducin alpha subunit.

It is specifically envisioned that, in addition to G proteins, many other proteins will be able to interact with the loop 3 region of the chimeric fusion protein of the present invention. A brief list of proteins known to interact with the loop 3 region of G protein-coupled receptor proteins includes protein 14-3-3 (J. Biol. Chem. 274(19):13462-9, 1999), which interacts with the alpha 2 adrenergic receptor; proteins Grb 2 and Nck (Biochemistry 37: 15726-36, 1998), which interact through SH3 interaction domains found on loop 3 of the dopamine D4 receptor; protein kinase A (Brain Research Bulletin 42:427-30, 1997), which phosphorylates cytoplasmic loop 3 of the alpha 1 adrenergic receptor; and rhodopsin kinase (J. Biol. Chem. 266: 12949-12955, 1991), which is activated by binding cytoplasmic loop 3 of rhodopsin).

It is reasonably expected that a bacteriorhodopsin/G protein-coupled receptor protein in which the intracellular loop 3 region of bacteriorhodopsin is replaced by the structurally analogous loop 3 region from a G protein-coupled receptor may interact with a protein that interacts with the loop 3 region of the native G protein-coupled receptor. One skilled in the art will appreciate that specific interaction between chimeric loop 3 polypeptides and proteins that interact with the loop 3 region of the native receptor can be evaluated by using specific assays. For example, the ability of protein kinase A to phosphorylate a bacteriorhodopsin/G protein-coupled receptor protein comprising a portion of the loop three region of the alpha 1 adrenergic receptor could be determined by measuring phosphorylation of the chimeric protein by protein kinase A using radiolabeled ATP as a substrate.

As demonstrated in the examples, chimeric bacteriorhodopsin/G protein-coupled receptor protein can be isolated intact in relatively large quantities. We expect that the availability of chimeric bacteriorhodopsin/G protein-coupled receptor protein will facilitate evaluation of potential pharmaceutical agents that can enhance or reduce activation of G proteins. Additional advantages of these fusion proteins is enhanced protein stability of the G protein-coupled receptor and opportunities for obtaining high resolution crystal structure information structure information, which may also facilitate drug development.

It is envisioned that the chimeric proteins of the present invention may be used in assays designed to evaluate interaction between potential therapeutic agents and the intracellular loop 3 of the G protein-coupled receptor. Any suitable means for evaluating the interaction of a potential pharmaceutical molecule with the intracellular loop 3 could be employed. One could evaluate the ability of the potential pharmaceutical agent to interact with intracellular loop 3 by incubating the agent with the protein under suitable conditions for a period of time sufficient to allow interaction to occur and evaluating the reaction mixture for the presence or absence of interaction. For example, interaction with loop three may alter the ability of the chimeric protein to promote GTP-GDP exchange, which could be measured as described in the examples. The ability of an agent to alter the activity of an enzyme that is activated by interaction with loop 3 or which has loop 3 as a substrate could be evaluated by measuring the agent's effect on the enzymatic reaction. Alternatively, the protein or test molecule could be detectably labeled to allow convenient detection of the interaction.

The following nonlimiting examples are intended to be purely illustrative.

### EXAMPLES

Bacterial Strains and Culture Conditions

Halobacterium salinarium MPK40 was grown as previously described (Krebs, et al. supra). This strain lacks a bacteriorhodopsin gene, and therefore does not make purple membranes. Briefly, a single colony of Halobacterium salinarium MPK40 was used to inoculate 5 ml of culture medium (4.2 M NaCl, 0.08 M MgSO₄, 0.01M trisodium citrate, 0.026 M KCl, yeast extract (3 g/l), tryptone (5 g/l)) and cultured for 4 days at 37° C. with 250 RPM shaking. For transformations, a 1-ml aliquot of the saturated starter culture was used to inoculate 50 ml of culture medium and grown for 20 hours at 37° C. with shaking to an optical density of 0.5 at A₆₆₀. For protein purifications, a 5-ml saturated starter culture was used to inoculate a 1.81 volume of culture medium, which was cultured as described above.

Construction of Bacteriorhodopsin Chimeras

The plasmid pMPK85 (Krebs et al. Biochemistry 38:9023-9030, 1999), a derivative of MPK62 (Krebs, et al., Proc. Natl. Acad. Sci. USA 90:1987-1991, 1993), was used to construct bacteriorhodopsin-G protein receptor chimeric proteins. The pMPK85 plasmid contains a DNA fragment comprising a bacteriorhodopsin gene (BOP) inserted into the BamHI site of the multiple cloning region, as well as mevinolin and ampicillin resistance markers (FIG. 1). The aforementioned publications by Krebs et al. are incorporated by reference in their entirety.

To obtain large quantities of the BOP gene, the gene was subcloned into an E. coli vector using standard molecular biological techniques. Briefly, the BOP gene was excised from the parent vector by digesting pMPK85 with BamHI restriction endonuclease. A BamHI fragment containing the BOP gene was ligated to BamHI-linearized PGEM 11ZF(+)vector (Promega Corp., Madison, Wis.), and the ligation mixture was used to transform Xl-1 Blue E. coli by electroporation. A transformant bearing a plasmid (designated pAHG) having the BOP insert was obtained. The plasmid was purified using a Qiagen plasmid miniprep kit, followed by agarose gel purification.

Purified pAHG plasmid was mutated using the Gene Editor kit (Promega Corp.) to create silent mutations that resulted in novel restriction sites. A primer having the sequence shown in SEQ ID NO:3 was used to create a BstXI site on transmembrane domain 4. A primer having the sequence shown in SEQ ID NO:4 was used to create a Bsu36I site on transmembrane domain 7. A mutated plasmid (designated pAHGB) containing both restriction sites was obtained.

To remove the region that encodes cytoplasmic loop 3 of BOP, pAHGB DNA was digested with BstXI and BsrGI. The BOP gene contains a native BsrGI site on transmembrane domain 5. The doubly-digested pAHGB was then treated with calf alkaline intestinal phosphatase and purified by agarose gel electrophoresis. Sequences encoding cytoplasmic loop 3 of bovine rhodopsin, adrenocortico-trophic hormone receptor, or adenosine A1 receptor were synthesized as described below (Generation of Loop 3 Inserts) and ligated to the large BstXI-BsrGI fragment from pAHGB to obtain pAHGBRI3,1; pAHGACTRI3,1; pAHGAAIAI3,1, respectively. Fragments containing sequences encoding the chimeric proteins comprising BOP and cytoplasmic loop 3 from a G protein receptor were ligated to BamHI-digested pMPK85 that was isolated from the BOP insert. The ligation mixture was introduced into E. coli by electroporation.

Transformants containing bacteriorhodopsin chimeric constructs having cytoplasmic loop 3 of bovine rhodopsin, adrenocorticotrophic hormone receptor, or adenosine A1 receptor were obtained and the plasmids were designated pMPKBRI3,1, pMPKACTRI3,1, and pMPKAAIAI3,1, respectively. Because this plasmid has a low copy number, a Qiagen midi plasmid prep kit was used to purify plasmid DNA from transformants.

The BamHI inserts from these plasmids were ligated to BamHI-linearized pAHG DNA, and the ligation mixture was used to transform H. salinarium MPK40 strain. The plasmid pAHG contains an insertion sequence ISH1 (SEQ ID NO:45) that allows the chimeric gene to integrate into the chromosome. Integration was confirmed by standard PCR analysis. DNA sequences of the chimeric protein coding regions were confirmed using the Big Dye sequencing system at the University of Wisconsin Biotech Center.

Generation of Cytoplasmic Loop 3 Inserts

Synthetic DNA oligonucleotides were obtained from Integrated DNA Technologies, Corp. The oligonucleotide sequences were designed so as to obtain maximum codon usage in H. salinarium, as discussed in Archea: A Laboratory Manual, Rob, F. T., et al., Cold Spring Harbor, Appendix 4, pp. 191-194, 1995).

The coding region for the bovine rhodospin cytoplasmic loop 3 from V230 to M283 of bovine rhodopsin chimera was obtained by combining SEQ ID NO: 3 and SEQ ID NO:4, which have a 20 base pair region of complementarity, heating the mixture to 98° C., and annealing by cooling the oligonucleotides in a Perkin-Elmer 480 thermocycler at a rate of 1.5° C./minute to 20° C.

| | | |
|---|---|---|
| SEQ ID NO:5 | Rhod I3, 1 TopA: | 5′ ATCCTGTACGTGCTGTTCTTCGGGTTCACCGTCAAGGAGGCGGCGGCGCAGCAGCAG |
| | | GAGTCGGCGACGACGCAGAAGGCGGAGAAGGAGG 3′ |
| and | | |
| SEQ ID NO:6 | Rhod I3, 1 BotA: | 5′ CGGGATACGCGGACCACAACACAACGGTAACGTTACGCAGTACTTTGAACGTGGATG |
| | | CGACCTCCATGCGCGTGACCTCCTTCTCCGCCTTCTGCG 3′ |

Sequences complementary to the BsrGI restriction site in transmembrane domain 5 and to the BstXI site in transmembrane domain 6 of the BOP gene were created by first treating the annealed oligonucleotide pairs with T4 DNA polymerase at 37° C. for 10 minutes and then amplifying the sequences using two different sets of primers (SEQ ID NO:7, 8, 9, and 10) and Pfu polymerase in a PCR reaction.

| | | |
|---|---|---|
| Set 1: | | |
| SEQ ID NO:7 | (NBBsrGITOPI): | 5′-GTACATCCTGTACGTGCTGTTCTTCG-3′ |
| SEQ ID NO:8 | (NBBstXIBOT2): | 5′-ACGACGGGATACGCGGACC-3′ |
| Set 2: | | |
| SEQ ID NO:9 | (NBBsrGITOP3): | 5′-ATCCTGTACGTGCTGTTCTTCG-3′ |
| SEQ ID NO:10 | (NBBstXIBOT4): | 5′-CGGGATACGCGGACC-3′ |

PCR products were purified by agarose gel electrophoresis, combined into a single PCR tube, heated to 98° C., and annealed by cooling. The constructs thus generated have BstXI and BsrGI compatible ends that allow ligation to the bacteriorhodopsin gene in the proper orientation.

The coding region of cytoplasmic loop 3 (A200 to G217) of adrenocorticotropic hormone receptor was prepared using synthetically prepared oligonucleotides SEQ ID NO:11 and SEQ ID NO:12, which have a 14-base pair region of complementarity.

| | | |
|---|---|---|
| SEQ ID NO:11 | (ACTRTOPA): | 5′ ATCCTGTACGTGCTGTTCTTCGGGTTCACCGCGCGCTCCCACACGCGCAAGATCTCC |
| | | ACGCTCCCGCGCGCGAACATGAAGGG 3′ |
| and | | |
| SEQ ID NO:12 | (ACTRBOTA): | 5′ CGGGATACGCGGACCACAACACAACGGTAACGTTACGCAGTACTTTGAACGTGGATG |
| | | CGACGCCCTTCATGTTCG 3′ |

These oligonucleotides were treated in the same manner as those used in creating the bacteriorhodopsin-bovine rhodopsin chimera.

A coding sequence for the loop 3 region (E202 to S235) of the adenosine A1 receptor was obtained by combining SEQ ID NO: 13 and SEQ ID NO:14, which have a 27 base pair region of complementarity, heating the mixture to 98° C., and annealing the oligonucleotides in a Perkin-Elmer 480 thermocycler.

| | | |
|---|---|---|
| SEQ ID NO:13 | (AAIATOPA): | 5′ GGGTTCACCGAGGTCTTCTACCTCATCCGCAAGCAGCTGGACAAGAAGGTCTCCGCG |
| | | TCCTCCGGCGACCCGCAGAAGTACTACGGCAAG 3′ |
| and | | |
| SEQ ID NO:14 | (AAIABOTA): | 5′ CACAACGGTAACGTTACGCAGTACTTTGAACGTGGATGCGACGGACTTCGCGATCTT |
| | | GAGCTCCTTGCCGTAGTACTTCTGCGGGTCGCC 3′ |

These oligos were treated in the same manner as those used in creating the bovine rhodopsin chimera.

An insert comprising a portion of the loop 3 region of the human beta2 adrenergic receptor adenosine was obtained by PCR amplification as described above using primer pair B2BOT900 (SEQ ID NO:48) and B2TOP1 (SEQ ID NO:52).

The PCR product thus obtained was amplified by PCR using the primers B2TOPA1 (SEQ ID NO:50) and B2TOPB1 (SEQ ID NO:53). This PCR product was then doubly digested with BSRG1 and BSTX1, and ligated to doubly digested pAHGB.

The coding sequence of the bacteriorhodopsin/beta 2 adrenergic receptor loop 3 is shown in SEQ ID NO:46 and SEQ ID NO:47. The beta2 adrenergic receptor portion of the loop 3 coding sequence is found between amino acid residues 12-73 of SEQ ID NO:47.

Construction of Additional Loop 3 Bovine Rhodopsin Chimeras

Nine additional bovine rhodopsin loop 3 inserts (designated 3A-3I) were obtained using the indicated oligonucleotides as detailed below. The amino acid sequences of the inserts are described by the corresponding sequences in native bovine rhodopsin. For example, insert 3A (InsQ225-M253) is 31 amino acid residues long and has the sequence corresponding to the amino acid residues 225 (glutamine)-253 (methionine) of bovine rhodopsin.

Inserts 3A-3D were constructed using SEQ ID NO: 15 and SEQ ID NO:16 in a first polymerase chain reaction to generate a single PCR product. The extension product was amplified by PCR using a second set of primers (shown below) to generate a final PCR product. These products were digested with BstXI and BsrGI, and ligated to vector DNA linearized by treatment with BstXI and BsrGI.

The following primer sets were used:

| | |
|---|---|
| InsQ225-M253 (3A): | |
| BOVI3_2top | (SEQ ID NO:15) |
| BOVI3_2BOT | (SEQ ID NO:16) |
| BOVI3_2 − 1TOBSRG1 | (SEQ ID NO:17) |
| BOVI3_2 + 1TO_BSTX1 | (SEQ ID NO:18) |
| InsY223-T251 (3B): | |
| BovI3_2top | (SEQ ID NO:15) |
| BOVI3_2BOT | (SEQ ID NO:16) |
| BOVI3_2 + 1TO_BSRG1 | (SEQ ID NO:19) |
| BOVI3_2 − 1BSTX1 | (SEQ ID NO:20) |
| InsY223-M253 (3C): | |
| BovI3_2top | (15) |
| BOVI3_2BOT | (SEQ ID NO:16) |
| BOVI3_2 + 1TO_BSTX1 | (SEQ ID NO:18) |
| BOVI3_2 + 1TO_BSRG1 | (SEQ ID NO:19) |
| InsQ225-T251 (3D): | |
| BovI3_2top | (SEQ ID NO:15) |
| BOVI3_2BOT | (SEQ ID NO:16) |
| BOVI3_2 − 1TOBSRG1 | (SEQ ID NO:17) |
| BOVI3_2 − 1BSTX1 | (SEQ ID NO:20) |
| InsG224-M253 (3E): | |
| BovI3_2top | (SEQ ID NO:15) |
| BOVI3_2BOT | (SEQ ID NO:16) |
| BOVBSRG1SITE | (SEQ ID NO:21) |
| BOVBSRG1NOSITE | (SEQ ID NO:22) |
| BOVI3_2 + 1TO_BSTX1 | (SEQ ID NO:18) |
| InsG224-T251 (3F): | |
| BovI3_2top | (SEQ ID NO:15) |
| BOVI3_2BOT | (SEQ ID NO:16) |
| BOVBSRG1SITE | (SEQ ID NO:21) |
| BOVBSRG1NOSITE | (SEQ ID NO:22) |
| BOVI3_2 − 1BSTX1 | (SEQ ID NO:20) |
| InsY223-R252 (3G): | |
| BovI3_2top | (SEQ ID NO:15) |
| BOVI3_2BOT | (SEQ ID NO:16) |
| BOVI3_2 + 1TO_BSRG1 | (SEQ ID NO:19) |
| BOVBSTX1CSITE | (SEQ ID NO:23) |
| BOVBSTX1NOCSITE | (SEQ ID NO:24) |
| InsQ225-R252 (3H): | |
| BovI3_2top | (SEQ ID NO:15) |
| BOVI3_2BOT | (SEQ ID NO:16) |
| BOVI3_2 − 1TOBSRG1 | (SEQ ID NO:17) |
| BOVBSTX1CSITE | (SEQ ID NO:23) |
| BOVBSTX1NOCSITE | (SEQ ID NO:24) |
| InsG224-R252 (3I): | |
| BovI3_2top | (SEQ ID NO:15) |
| BOVI3_2BOT | (SEQ ID NO:16) |
| BOVBSRG1SITE | (SEQ ID NO:21) |
| BOVBSRG1NOSITE | (SEQ ID NO:22) |
| BOVBSTX1CSITE | (SEQ ID NO:23) |
| BOVBSTX1NOCSITE | (SEQ ID NO:24) |

For the construction of inserts 3E-3I, the following series of PCR reactions were performed. SEQ ID NO:15 and SEQ ID NO:16 were annealed and amplified by PCR to generate a single PCR product. This PCR product was PCR-amplified two times using primers having either one site (BsrGI or BstXI) or both sides of the insert with both the ***SITE and ***NOSITE primers. Both of these second PCR products were digested with either BsrGI or BstXI, or neither. After digestion the PCR products were added together and heated and reannealed to generate a site complementary to either the BsrGI or BstXI site. This reanealed insert was ligated into pAHD digested with BstXI and BsrGI.

By way of illustration, the 3H chimera was generated using the indicated primers as described below.

| | |
|---|---|
| InsQ225-R252 (3H): | |
| BovI3_2top | (SEQ ID NO:15) |
| BOVI3_2BOT | (SEQ ID NO:16) |
| BOVI3_2 − 1TOBSRG1 | (SEQ ID NO:17) |
| BOVBSTX1CSITE | (SEQ ID NO:23) |
| BOVBSTX1NOCSITE | (SEQ ID NO:24) |

After generating the first PCR product for this insert using SEQ ID NO:15 and SEQ ID NO:16, the PCR product was amplified in separate PCR reactions using the following sets of primers:

| | |
|---|---|
| BOVI3_2 − 1TOBSRG1 | (SEQ ID NO:17) |
| BOVBSTX1CSITE | (SEQ ID NO:23) |
| and | |
| BOVI3_2 − 1TOBSRG1 | (SEQ ID NO:17) |
| BOVBSTX1NOCSITE | (SEQ ID NO:24) |

to obtain two second PCR products. The two second products were digested with BsrGI. The BsrGI-digested PCR products were combined, heated, and annealed, and then ligated directly to linearized pAHG vector DNA having compatible ends. This approach was followed for inserts 3E-3I.

The coding sequence and amino acid sequence of each chimeric loop 3 region, including the N-terminal threonine residue and the C-terminal valine residue of bacteriorhodopsin, are provided. The coding sequences for inserts 3A, 3B, 3C, 3D, 3E, 3F, 3G, 3H, and 3I correspond to SEQ ID NO: 25, 27, 29, 31, 33, 35, 37, 39, and 41, respectively; the corresponding amino acid sequences of the loop region inserts are presented in SEQ ID NO: 26, 28, 30, 32, 34, 36, 38, 40, and 42.

Transformation of H. salinarium

Transformation was carried out as previously described (Krebs, et al. supra). Briefly, a 10 ml aliquot of an MPK40 culture (OD₆₆₀=0.5) was centrifuged at 3,000× g for 10 minutes. The cell pellet was resuspended in 1 ml of spheroplasting solution (2M NaCl, 27 mM KCl, 50 mM Tris-Cl, pH 8.5, 15% w/v sucrose). Aliquots (0.2 ml) of cells resuspended in spheroplasting solution were gently agitated with 10 uL of EDTA-spheroplasting solution (spheroplasting solution containing 0.5M EDTA). Plasmid DNA (1.5 ug) in a volume of approximately 3 uL was diluted with 13.5 uL of spheroplasting solution. The DNA sample was mixed with the 0.2 ml cells resuspended in spheroplasting solution and the mixture was incubated at room temperature for 5 minutes. A 225-uL aliquot of PEG-spheroplasting solution (60% (w/v) PEG 600 to spheroplasting solution) was added to each sample. After 20 minutes, 5 ml of CM sucrose (15% w/v of sucrose in culture medium) was added to the mixture, and the sample was centrifuged for 20 minutes at 5,000× g. The pellet was resuspended in 10 ml of CM sucrose and incubated for 16 hours at 37° C. on a shaker at 250 RPM. Aliquots of 100 uL of the sample were plated on Mevinolin-complemented complemented agar culture medium plates. Transformants were transferred to 1 ml of culture media, diluted 100 fold, and 100 ul aliquots were plated on CM plates.

Stable transformants having the bacteriorhodopsin G-protein coupled protein gene integrated into the chromosome were evaluated for the production of a purple pigment by placing the CM plates under white lights for 48-72 hours, after which time purple colonies were observable. Purple pigment production indicates that the bacteriorhodopsin is able to bind retinal. PCR analysis was used to confirm chimera gene insertion. Transformants comprising, bacteriorhodopsin/bovine rhodopsin bacteriorhodopsin/adrenocorticotropin releasing hormone receptor, or the bacteriorhodopsin/human beta2 adrenergic receptor appeared purple, whereas transformants comprising the bacteriorhodopsin/A-1 adenosine chimera appeared orange. These results indicate that the stable transformants comprising the bacteriorhodopsin/bovine rhodopsin chimera or the bacteriorhodopsin/adrenocortico-tropin releasing hormone receptor chimera form purple membranes having a chromophore similar to that of bacteriorhodopsin.

Purification of Bacteriorhodopsin/G-Protein-Coupled Receptor Chimeric Protein

Chimeric bacteriorhodopsin was purified from a transformant comprising the bacteriorhodopsin/bovine rhodopsin chimera in essentially the same way that native bacteriorhodopsin is purified from archaebacterial species (4). Briefly, a single purple colony was used to inoculate 5 ml of culture media and cultured for 4 days at 37° C. with shaking at 250 RPM. This culture was used to inoculate 1.8 L of culture medium, and the culture was grown for 5 days in an illuminated shaker (wavelength 570 nM) at 40° C. and 250 RPM. Cells were pelleted by centrifugation at 7,000× g for 30 minutes and resusupended in 18 ml of water, and treated with 500 units of DNaseI. This preparation was dialyzed against water for 10 hours with constant stirring. The dialysate was then centrifuged at 23,000× g for 40 minutes. The pellets were resuspended in 2 ml of 5 mM NaCl, and placed over continuous sucrose gradients (30-58%). The gradients were centrifuged in a swinging bucket rotor at 27,000× g for 17 hours at 15° C. A purple band that migrated near the bottom of the centrifuge tube was collected. Typically, this purification procedure yields between 5 and 10 milligrams of pure protein.

Characterization of Bacteriorhodopsin-Rhodopsin Chimera

The protein was characterized by ultraviolet and visible absorbance spectrophotometry (UV/VIS), circulator dichromism (CD), and SDS-PAGE. The results of SDS-PAGE size estimates were consistent with the predicted molecular weight based on the deduced amino acid sequence.

A comparison of the UV/vis spectra from the bacterio-rhodopsin/bovine rhodopsin chimeric protein spectra reveals very similar spectra, suggesting that the chimeric protein has the same chromophore (retinal) as the native bacteriorhodopsin that the chimeras may have the proper lattice formation.

The ability of the chimeric protein to promote GTP-GDP exchange was evaluated for each of three BR-rhodopsin loop 3 chimeras by evaluating the effect of the chimera on the rate of GDP-GTP exchange on transducin using a GTPyS uptake assay, as described below.

Chimeric or bacteriorhodopsin protein (1.7 uM) was incubated with 0.8 uM holotransducin with 10 nM ³⁵S radiolabeled GTPyS in buffer (20 mM Hepes (pH 7.5), 5 mM MgCl₂, 1 mM DTT, 0.1 mM EDTA, and 20 mM NaCl). The reaction was incubated at 30° C. for 5, 10, 15 or 30 minutes. The reaction was terminated by transferring aliquots of the reaction mixture to nitrocellulose filters (0.45 um) prewetted with wash buffer (10 mM Tris (pH 7.5), 5 mM MgCl₂, 100 mM NaC1, 0.1 mM EDTA) and placed on a vacuum manifold. The filters were then washed with 8 ml of wash buffer. The filters were then placed overnight in scintillation vials filled with 20 ml of scintillation cocktail. The activity of the vials was counted by a Liquid Scintillation Counter. For the BR-Rhodopsin loop 3 chimeras, in which the rhodopsin intracellular loop 3 replaced the native BR intracellular loop 3 and was fused from T157 on BR TM 5 to V167 on BR TM 6, activity was determined by an increased uptake of GTPS over basal exchange by transducin alone.

| Specific counts (Lattice Form) | (at 30 min) |
|---|---|
| Rhodopsin Insert Y223 to M253 (3C) | 262331 |
| Rhodopsin Insert Y223 to R252 (3G) | 182530 |
| Rhodopsin Insert Q225 to R252 (3H) | 193374 |
| BR | 117859 |
| Basal Exchange of Transducin | 164750 |

| Activation (Fold increase) | over BR | over Basal |
|---|---|---|
| Rhodopsin Insert Y223 to M253 (3C) | 2.22 | 1.6 |
| Rhodopsin Insert Y223 to R252 (3G) | 1.55 | 1.1 |
| Rhodopsin Insert Q225 to R252 (3H) | 1.6 | 1.17 |

A time course study was next performed for Rhodopsin Insert Y223 to M253 (3C)

| Specific counts (Lattice Form) | 5 | 10 | 15 min |
|---|---|---|---|
| Rhodopsin Insert | 26273 | 51423 | 66566 |
| (Y223 to M253(3C)) | | | |
| Basal Exchange | 16825 | 26325 | 33653 |

| Activation by 3C (×100%) | Over Basal Exchange |
|---|---|
| 5 Minutes | 1.56 |
| 10 Minutes | 1.95 |
| 15 Minutes | 1.97 |

Additionally, the GTPyS exchange assay was repeated in the presence of varying concentrations of either a peptide that has been shown to inhibit transducin activation by rhodopsin (“high affinity analog”), or a random peptide.

The “high affinity analog” is the C-terminal portion of the transducin alpha subunit that competitively inhibits binding of transducin to G protein-coupled receptor protein. The high affinity analog has the sequence V-L-E-D-L-K-S-C-G-L-F-G (SEQ ID NO:43) (J. Biol. Chem. 271 pp. 361-366, 1996).

The random peptide consists of the sequence S-S-V-F-L-V-V-D-R-S-R (SEQ ID NO:44).

The addition of the high affinity analog at a final concentration of 8 uM gave the following results.

| Specific counts (Lattice Form) | 10 Minutes |
|---|---|
| Rhodopsin Insert Y223 to M253 (3C) | 51423 |
| Basal Exchange | 26325 |
| 3C + 8 uM HIGH AFFINITY ANALOG | 42455 |
| Basal Exchange + 8 uM H.A.A. | 23154 |
| 3C + 8 uM Random Peptide | 47516 |
| Basal Exchange + 8 uM Random Peptide | 23868 |

% Inhibition of GTPγS Uptake (Controlled for Peptide
inhibition of Basal Exchange)

| | |
|---|---|
| 8 uM High affinity analog | 23% |
| 8 uM Random peptide | 5.8% |

The second inhibition experiment used 50 uM of the high affinity analog.

| SPECIFIC COUNTS: (Lattice Form) | 10 Minutes |
|---|---|
| Rhodopsin Insert Y223 to M253 (3C) | 93448 |
| Basal Exchange | 49988 |
| 3C + 50 uM H.A.A. | 46892 |
| Basal Exchange + 50 uM H.A.A. | 33888 |
| 3C + 50 uM Random Peptide | 35568 |

% Inhibition of GTPγS Uptake (Controlled for Peptide
inhibition of Basal Exchange)

| | |
|---|---|
| 50 uM HIGH AFFINITY ANALOG | 70% |
| 50 uM Random peptide | 30% |

The next set of experiments was designed to measure the effects of GDP concentration on the basal exchange of GTPγS by holotransducin.

| Specific counts (Lattice Form) | 10 Minutes |
|---|---|
| Rhodopsin Insert Y223 to M253 100 uM GDP | 12577 |
| Basal Exchange + 100 uM GDP | 7502 |
| Basal Exchange + 50 uM GDP | 11061 |
| Basal Exchange + 10 uM GDP | 22885 |
| Basal Exchange + 0 uM GDP | 51307 |

| | |
|---|---|
| Activation (3C) (×10°) | over Basal Exchange |

| | |
|---|---|
| Rhodopsin Insert Y223 to M253 100 μM GDP | 1.67 |

The following experiments repeated the study of GTPγS exchange with a new culture of the BR-Rhodopsin chimera for Rhodopsin Insert Y223 (3C) as well as three new chimeras.

| Specific counts (Lattice Form) | 10 Minutes |
|---|---|
| Rhodopsin Insert Y223 to T251 (3B) | 62759 |
| Rhodopsin Insert Y223 to M253 (3C) | 77125 |
| Rhodopsin Insert Q225 to T251 (3D) | 53448 |
| Rhodopsin Insert G224 to M253 (3E) | 78433 |
| Basal Exchange | 53565 |

| Activation (×100%) | Over Basal Exchange |
|---|---|
| Rhodopsin Insert Y223 to T251 (3B) | 1.17 |
| Rhodopsin Insert Y223 to M253 (3C) | 1.44 |
| Rhodopsin Insert Q225 to T251 (3D) | 1.0 |
| Rhodopsin Insert G224 to M253 (3E) | 1.46 |

The present invention is not limited to the exemplified embodiments, but is intended to encompass all such modifications and variations as come within the scope of the following claims.

1. A chimeric fusion protein comprising a bacteriorhodopsin protein amino acid sequence in which at least a portion of the protein is replaced with the structurally analogous region of a G protein-coupled receptor protein.

2. The protein of claim 1, wherein the protein comprises substantially all of the amino acid sequence of bacteriorhodopsin except the intracellular loop 3 domain, wherein the intracellular loop 3 domain of bacteriorhodopsin is replaced by at least a portion of the intracellular loop 3 domain of a G protein-coupled receptor protein.

3. The chimeric protein of claim 2, wherein the intracellular loop 3 domain region corresponding to amino acid residues 171-179 of SEQ ID NO:2 is replaced with at least a portion of the intracellular loop 3 domain of a G protein-coupled receptor protein.

4. The chimeric protein of claim 2, wherein the protein is able to alter the rate of GTP-GDP exchange on a G protein in vitro.

5. The chimeric protein of claim 4, wherein the rate of GTP-GDP exchange is increased.

6. A polynucleotide sequence encoding the chimeric fusion protein of claim 1.

7. A genetic construct comprising the polynucleotide sequence of claim 6, the polynucleotide sequence operably connected to a promoter sequence.

8. An archaebacterium comprising the genetic construct of claim 7, wherein the polynucleotide sequence of the construct is expressible in the archaebacterium.

9. The archaebacterium of claim 8, wherein the archaebacterium is characterized by reduced expression of wild type bacteriorhodopsin.

10. The archaebacterium of claim 8, wherein the genetic construct is integrated into the archaebacterium chromosome.

11. A method of producing a bacteriorhodopsin/G protein-coupled receptor chimeric fusion protein comprising the step culturing an archaebacterium comprising a genetic construct having a polynucleotide sequence that encodes a chimeric fusion protein having bacteriorhodopsin protein amino acid sequence in which at least a portion of the protein is replaced with the structurally analogous region of a G protein-coupled receptor protein, the polynucleotide sequence operably connected to a promoter sequence functional in the archaebacterium, wherein the polynucleotide sequence of the construct is expressible in the archaebacterium, under suitable conditions and for a period of time sufficient to allow expression of the chimeric fusion protein.

12. The method of claim 11, further comprising the step of partially purifying the chimeric fusion protein.

13. A method of testing a molecule for its ability to interact with the intracellular loop 3 of a G protein-coupled receptor comprising the steps of:
(a) reacting a chimeric fusion protein of comprising a substantially all of the bacteriorhodopsin protein amino acid sequence amino acid sequence except the intracellular loop 3 domain, wherein the intracellular loop 3 domain of bacteriorhodopsin is replaced by at least a portion of the intracellular loop 3 domain of a G protein-coupled receptor protein with a test molecule under suitable reaction conditions for a period of time sufficient to allow interaction between the molecule and the protein; and
(b) detecting presence or absence of interaction between the protein and the test molecule in the reaction mixture.

14. The method of claim 13, wherein the chimeric fusion protein of step (a) is able to promote GTP-GDP exchange on a G protein in vitro, and wherein the detecting step (b) includes an in vitro GTP-GDP exchange assay.


SEQUENCE LISTING (53 sequences)

SEQ ID NO: 1 - DNA, 1626 bp, Halobacterium salinarium
  CDS (394)..(1182)
ggatccgacgtgaagatggggctcccgatgggtgcaaccgtgaagtccgtcacggctgcgtcacgacaggagccgaccagcgacacccagaaggtgcgaacggttgagtgccgcaacgatcacgagtttttcgtgcgcttcgagtggtaacacgcgtgcacgcatcgacttcaccgcgggtgtttcgacgccagccggccgttgaaccagcaggcagcgggcatttcacagccgctgtggcccacacactcggtggggtgcgctattttggtatggtttggaatccgcgtgtcggctccgtgtctgacggttcatcggtctaaattccgtcacgagcgtaccatactgattgggtcgtagagttacacacatatcctcgttaggtactgttgcatgttggagttattgccaacagcagtggagggggtatcgcaggcccagatcaccggacgtccggagtggatctggctagcgctcggtacggcgctaatgggactcgggacgctctatttcctcgtgaaagggatgggcgtctcggacccagatgcaaagaaattctacgccatcacgacgctcgtcccagccatcgcgttcacgatgtacctctcgatgctgctggggtatggcctcacaatggtaccgttcggtggggagcagaaccccatctactgggcgcggtacgctgactggctgttcaccacgccgctgttgttgttagacctcgcgttgctcgttgacgcggatcagggaacgatccttgcgctcgtcggtgccgacggcatcatgatcgggaccggcctggtcggcgcactgacgaaggtctactcgtaccgcttcgtgtggtgggcgatcagcaccgcagcgatgctgtacatcctgtacgtgctgttcttcgggttcacctcgaaggccgaaagcatgcgccccgaggtcgcatccacgttcaaagtactgcgtaacgttaccgttgtgttgtggtccgcgtatcccgtcgtgtggctgatcggcagcgaaggtgcgggaatcgtgccgctgaacatcgagacgctgctgttcatggtgcttgacgtgagcgcgaaggtcggcttcgggctcatcctcctgcgcagtcgtgcgatcttcggcgaagccgaagcgccggagccgtccgccggcgacggcgcggccgcgaccagcgactgatcgcacacgcaggacagccccacaaccggcgcggcttttcaacgacacacgatgagtcccccactcggtcttgtactcgcacgatcgcgcgacgacggcgacgccgacggcgactttccagcgtcgctcaacaggctggctgtcgtcgcgctcgctggtgcggctctcgtcggtgcggcgggtctgttcgccgtgccgttcctgcggtcgttcggcatgacgttttgggaagcgttcaccgttgttggtgtctccgagttcgtctcggccatcgtggcggccctcgcgggctaccacctctacaccacgcccgacgactagcagggcccgctggcgagccatcactcccgctgtggcgaggcgacggccgttctgtaccgctaccgccggcccggagtccgggacatcggcggggcgatgcgcatcgaacggtcacccggatcc

SEQ ID NO: 2 - protein, 262 aa, Halobacterium salinarium
MLELLPTAVEGVSQAQITGRPEWIWLALGTALMGLGTLYFLVKGMGVSDPDAKKFYAITTLVPAIAFTMYLSMLLGYGLTMVPFGGEQNPIYWARYADWLFTTPLLLLDLALLVDADQGTILALVGADGIMIGTGLVGALTKVYSYRFVWWAISTAAMLYILYVLFFGFTSKAESMRPEVASTFKVLRNVTVVLWSAYPVVWLIGSEGAGIVPLNIETLLFMVLDVSAKVGFGLILLRSRAIFGEAEAPEPSAGDGAAATSD

SEQ ID NO: 3 - DNA, 20 bp, Artificial Sequence
  Description of Artificial Sequence oligonucleotide primer
cgcgtatccagtcgtgtggc

SEQ ID NO: 4 - DNA, 20 bp, Artificial Sequence
  Description of Artificial Sequence oligonucleotide primer
cctcctgaggagtcgtgcga

SEQ ID NO: 5 - DNA, 91 bp, Artificial Sequence
  Description of Artificial Sequence oligonucleotide primer
atcctgtacgtgctgttcttcgggttcaccgtcaaggaggcggcggcgcagcagcaggagtcggcgacgacgcagaaggcggagaaggagg

SEQ ID NO: 6 - DNA, 96 bp, Artificial Sequence
  Description of Artificial Sequence oligonucleotide primer
cgggatacgcggaccacaacacaacggtaacgttacgcagtactttgaacgtggatgcgacctccatgcgcgtgacctccttctccgccttctgcg

SEQ ID NO: 7 - DNA, 26 bp, Artificial Sequence
  Description of Artificial Sequence oligonucleotide primer
gtacatcctgtacgtgctgttcttcg

SEQ ID NO: 8 - DNA, 19 bp, Artificial Sequence
  Description of Artificial Sequence oligonucleotide primer
acgacgggatacgcggacc

SEQ ID NO: 9 - DNA, 22 bp, Artificial Sequence
  Description of Artificial Sequence oligonucleotide primer
atcctgtacgtgctgttcttcg

SEQ ID NO: 10 - DNA, 15 bp, Artificial Sequence
  Description of Artificial Sequence oligonucleotide primer
cgggatacgcggacc

SEQ ID NO: 11 - DNA, 83 bp, Artificial Sequence
  Description of Artificial Sequence oligonucleotide primer
atcctgtacgtgctgttcttcgggttcaccgcgcgctcccacacgcgcaagatctccacgctcccgcgcgcgaacatgaaggg

SEQ ID NO: 12 - DNA, 75 bp, Artificial Sequence
  Description of Artificial Sequence oligonucleotide primer
cgggatacgcggaccacaacacaacggtaacgttacgcagtactttgaacgtggatgcgacgcccttcatgttcg

SEQ ID NO: 13 - DNA, 89 bp, Artificial Sequence
  Description of Artificial Sequence oligonucleotide primer
gggttcaccgaggtcttctacctcatccgcaagcagctgacaagaaggtctccgcgtcctccggcgacccgcagaagtactacggcaag

SEQ ID NO: 14 - DNA, 90 bp, Artificial Sequence
  Description of Artificial Sequence oligonucleotide primer
cacaacggtaacgttacgcagtactttgaacgtggatgcgacggacttcgcgatcttgagctccttgccgtagtacttctgcgggtcgcc

SEQ ID NO: 15 - DNA, 84 bp, Artificial Sequence
  Description of Artificial Sequence oligonucleotide primer
gggttcaccggccagctcgtcttcacggtcaaggaggcggcggcgcagcagcaggagtcggcgacgacgcagaaggcggagaag

SEQ ID NO: 16 - DNA, 90 bp, Artificial Sequence
  Description of Artificial Sequence oligonucleotide primer
ggaccacaacacaacggtaacgttacgcagtactttgaacgtggatgcgacgcggctgacctccttctccgccttctgcgtcgtcgccga

SEQ ID NO: 17 - DNA, 68 bp, Artificial Sequence
  Description of Artificial Sequence oligonucleotide primer
tttgtacatgtacatcctgtacgtgctgttcttcgggttcacccagctcgtcttcacggtcaaggagg

SEQ ID NO: 18 - DNA, 100 bp, Artificial Sequence
  Description of Artificial Sequence oligonucleotide primer
gctgccgatcagccacacgactggatacgcggaccacaacacaacggtaacgttacgcagtactttgaacgtggatgcgaccatgcgcgtgacctccttc

SEQ ID NO: 19 - DNA, 74 bp, Artificial Sequence
  Description of Artificial Sequence oligonucleotide primer
tttgtacatgtacatcctgtacgtgctgttcttcgggttcacctacggccagctcgtcttcacggtcaaggagg

SEQ ID NO: 20 - DNA, 100 bp, Artificial Sequence
  Description of Artificial Sequence oligonucleotide primer
gctgccgatcagccacacgactggatacgcggaccacaacacaacggtaacgttacgcagtactttgaacgtggatgcgaccgtgacctccttctccgcc

SEQ ID NO: 21 - DNA, 37 bp, Artificial Sequence
  Description of Artificial Sequence oligonucleotide primer
gtacatcctgtacgtgctgttcttcgggttcaccggc

SEQ ID NO: 22 - DNA, 33 bp, Artificial Sequence
  Description of Artificial Sequence oligonucleotide primer
atcctgtacgtgctgttcttcgggttcaccggc

SEQ ID NO: 23 - DNA, 31 bp, Artificial Sequence
  Description of Artificial Sequence oligonucleotide primer
acgacgggatacgcggaccacaacacaacgg

SEQ ID NO: 24 - DNA, 27 bp, Artificial Sequence
  Description of Artificial Sequence oligonucleotide primer
cgggatacgcggaccacaacacaacgg

SEQ ID NO: 25 - DNA, 93 bp, Artificial Sequence
  Description of Artificial Sequence oligonucleotide primer chimeric loop 3 sequence
acccagctcgtcttcacggtcaaggaggcggcggcgcagcagcaggagtcggcgacgacgcagaaggcggagaaggaggtcacgcgcatggtc

SEQ ID NO: 26 - protein, 31 aa, Artificial Sequence
  Description of Artificial Sequencechimeric loop 3 sequence
TQLVFTVKEAAAQQQESATTQKAEKEVTRMV

SEQ ID NO: 27 - DNA, 93 bp, Artificial Sequence
  CDS (1)..(93)
  Description of Artificial Sequence oligonucleotide primer chimeric loop 3 sequence
acctacggccagctcgtcttcacggtcaaggaggcggcggcgcagcagcaggagtcggcgacgacgcagaaggcggagaaggaggtcacggtc

SEQ ID NO: 28 - protein, 31 aa, Artificial Sequence
  Description of Artificial Sequencechimeric loop 3 sequence
TYGQLVFTVKEAAAQQQESATTQKAEKEVTV

SEQ ID NO: 29 - DNA, 99 bp, Artificial Sequence
  Description of Artificial Sequence oligonucleotide primer chimeric loop 3 sequence
acctacggccagctcgtcttcacggtcaaggaggcggcggcgcagcagcaggagtcggcgacgacgcagaaggcggagaaggaggtcacgcgcatggtc

SEQ ID NO: 30 - protein, 33 aa, Artificial Sequence
  Description of Artificial Sequencechimeric loop 3 sequence
TYGQLVFTVKEAAAQQQESATTQKAEKEVTRMV

SEQ ID NO: 31 - DNA, 87 bp, Artificial Sequence
  CDS (1)..(87)
  Description of Artificial Sequence oligonucleotide primer chimeric loop 3 sequence
acccagctcgtcttcacggtcaaggaggcggcggcgcagcagcaggagtcggcgacgacgcagaaggcggagaaggaggtcacggtc

SEQ ID NO: 32 - protein, 29 aa, Artificial Sequence
  Description of Artificial Sequencechimeric loop 3 sequence
TQLVFTVKEAAAQQQESATTQKAEKEVTV

SEQ ID NO: 33 - DNA, 96 bp, Artificial Sequence
  CDS (1)..(96)
  Description of Artificial Sequence oligonucleotide primer chimeric loop 3 sequence
accggccagctcgtcttcacggtcaaggaggcggcggcgcagcagcaggagtcggcgacgacgcagaaggcggagaaggaggtcacgcgcatggtc

SEQ ID NO: 34 - protein, 32 aa, Artificial Sequence
  Description of Artificial Sequencechimeric loop 3 sequence
TGQLVFTVKEAAAQQQESATTQKAEKEVTRMV

SEQ ID NO: 35 - DNA, 90 bp, Artificial Sequence
  CDS (1)..(90)
  Description of Artificial Sequence oligonucleotide primer chimeric loop 3 sequence
accggccagctcgtcttcacggtcaaggaggcggcggcgcagcagcaggagtcggcgacgacgcagaaggcggagaaggaggtcacggtc

SEQ ID NO: 36 - protein, 30 aa, Artificial Sequence
  Description of Artificial Sequencechimeric loop 3 sequence
TGQLVFTVKEAAAQQQESATTQKAEKEVTV

SEQ ID NO: 37 - DNA, 96 bp, Artificial Sequence
  CDS (1)..(96)
  Description of Artificial Sequence oligonucleotide primer chimeric loop 3 sequence
acctacggccagctcgtcttcacggtcaaggaggcggcggcgcagcagcaggagtcggcgacgacgcagaaggcggagaaggaggtcacgcgcgtc

SEQ ID NO: 38 - protein, 32 aa, Artificial Sequence
  Description of Artificial Sequencechimeric loop 3 sequence
TYGQLVFTVKEAAAQQQESATTQKAEKEVTRV

SEQ ID NO: 39 - DNA, 90 bp, Artificial Sequence
  CDS (1)..(90)
  Description of Artificial Sequence oligonucleotide primer chimeric loop 3 sequence
acccagctcgtcttcacggtcaaggaggcggcggcgcagcagcaggagtcggcgacgacgcagaaggcggagaaggaggtcacgcgcgtc

SEQ ID NO: 40 - protein, 30 aa, Artificial Sequence
  Description of Artificial Sequencechimeric loop 3 sequence
TQLVFTVKEAAAQQQESATTQKAEKEVTRV

SEQ ID NO: 41 - DNA, 93 bp, Artificial Sequence
  CDS (1)..(93)
  Description of Artificial Sequence oligonucleotide primer chimeric loop 3 sequence
accggccagctcgtcttcacggtcaaggaggcggcggcgcagcagcaggagtcggcgacgacgcagaaggcggagaaggaggtcacgcgcgtc

SEQ ID NO: 42 - protein, 31 aa, Artificial Sequence
  Description of Artificial Sequencechimeric loop 3 sequence
TGQLVFTVKEAAAQQQESATTQKAEKEVTRV

SEQ ID NO: 43 - protein, 12 aa, Artificial Sequence
  Description of Artificial Sequence high affinity analog
VLEDLKSCGLFG

SEQ ID NO: 44 - protein, 11 aa, Artificial Sequence
  Description of Artificial Sequencerandom peptide
SSVFLVVDRSR

SEQ ID NO: 45 - DNA, 91 bp, Halobacterium salinarium
cctgcagggtcgctggactcatccacctcagcattcaccctgctctttggtgtgctactcgttctatgacaccctcggaccaatactggct

SEQ ID NO: 46 - DNA, 266 bp, human
  CDS (2)..(265)
gtacatcctgtacgtgctgttcttcgggttcacccgcgtcttccaggaggcgaagcgccagctccagaagatcgacaagtccgagggccgcttccacgtccagaacctctcccaggtcgagcaggacggccgcaccggccacggcctccgccgctcctccaagttctgcctcaaggagcacaaggcgctcaagaccctcgaggtcgcatccacgttcaaagtactgcgtaacgttaccgttgtgttgtggtccgcgtatccctcgt

SEQ ID NO: 47 - protein, 88 aa, human
YILYVLFFGFTRVFQEAKRQLQKIDKSEGRFHVQNLSQVEQDGRTGHGLRRSSKFCLKEHKALKTLEVASTFKVLRNVTVVLWSAYPS

SEQ ID NO: 48 - DNA, 90 bp, Artificial Sequence
  Description of Artificial Sequence oligonucleotide primer
ggatgcgacctcgagggtcttgagcgccttgtgctccttgaggcagaacttggaggagcggcggaggccgtggccggtgcggccgtcctg

SEQ ID NO: 49 - DNA, 89 bp, Artificial Sequence
  Description of Artificial Sequence oligonucleotide primer
ggatacgcggaccacaacacaacggtaacgttacgcagtactttgaacgtggatgcgacctcgagggtcttgagcgccttgtgctcctt

SEQ ID NO: 50 - DNA, 24 bp, Artificial Sequence
  Description of Artificial Sequence oligonucleotide primer
tgtacatgtacatcctgtacgtgc

SEQ ID NO: 51 - DNA, 90 bp, Artificial Sequence
  Description of Artificial Sequence oligonucleotide primer
atcctgtacgtgctgttcttcgggttcacccgcgtcttccaggaggcgaagcgccagctccagaagatcgacaagtccgagggccgcttc

SEQ ID NO: 52 - DNA, 90 bp, Artificial Sequence
  Description of Artificial Sequence oligonucleotide primer
aagcgccagctccagaagatcgacaagtccgagggccgcttccacgtccagaacctctcccaggtcgagcaggacggccgcaccggccac

SEQ ID NO: 53 - DNA, 35 bp, Artificial Sequence
  Description of Artificial Sequence oligonucleotide primer
gctgccgatcagccacacgactggatacgcggacc